(19) United States
(12) Patent Application Publication
(10) Pub. No.: US 2009/0110724 A1
(43) Pub. Date: Apr. 30, 2009

(54) COMPOSITIONS AND METHODS FOR TREATMENT OF PAIN

(75) Inventor: John A. Giordano, West Orange, NJ (US)

Correspondence Address: DON J. PELTO;Sheppard, Mullin, Richter & Hampton LLP, 1300 I STREET, NW, 11TH FLOOR EAST, WASHINGTON, DC, 20005, US

(73) Assignee: Everett Laboratories, Inc.

(21) Appl. No.: 11/932,237

(22) Filed: Oct. 31, 2007

(51) Int. Cl. A61K 31/19; A61K 31/515; A61K 31/522; A61K 9/64; A61P 43/00

(52) U.S. Cl. 424/456; 514/263.3; 514/270; 514/568

(57) ABSTRACT

The present invention relates to compositions and methods for treatment of various forms of pain. Specifically, the method involves administering to a patient a composition comprising butalbital, acetaminophen and caffeine in the gelcap dosage form, to treat and/or alleviate the occurrence or negative effects of various forms of headaches.

### FIELD OF THE INVENTION

The present invention relates to compositions that may be in a form of a gel caplet (gelcap), comprising a barbiturate, a non-salicylate analgesic/antipyretic, and an analgesic adjunct, and methods for administering these compositions for treatment of patients suffering from various forms of pain.

### BACKGROUND OF THE INVENTION

Pain is an unpleasant sensation with a wide range in severity that can be localized or prevalent throughout the body. The Merck Manual of Medical Information (M. Beers et al., 2^{nd} Home ed., Merck Research Laboratories, 2003). Pain is affected by nerve stimulation that carries impulses to the brain and is a symptom of an underlying disease, disorder, or physical injury. Id.

It is estimated that 75 million Americans currently suffer with pain. NATIONAL CTR. FOR HEALTH STATISTICS, HEALTH, UNITED STATES, 2006 WITH CHARTBOOK ON TRENDS IN THE HEALTH OF AMERICANS 68-71 (2006). Pain can be classified into two groups: acute pain or chronic pain. According to the National Centers for Health Statistics, more than 50 million Americans experience chronic pain. Id. Chronic pain is a persisting or reoccurring pain that can last for months and even years. It is more common for chronic pain to be due to a disorder such as sickle cell anemia or a serious physical injury.

Alternatively, acute pain tends to come on quickly, but subsides after a short time. Acute pain serves a useful purpose by alerting humans of a physiological hazard or a need for treatment. Therefore, acute pain tends to be caused from a sudden physical injury or infection.

Clinical complaints due to pain come in many varieties. Such complaints may be due to such ailments as arthritis, back pain, neuropathy, or a headache.

A headache, also known as cephalalgia, ranks amongst the most common clinical pain complaints and can be caused from a wide variety of physiological effects. Such causes range from hormonal changes, muscle tension in the back of the neck, or from dehydration. Because headaches come in many forms, they may be treated or prevented by various methods. These methods may include maintaining a healthy lifestyle, reducing stress or making use of various medications. Such medications can be antianxiety drugs, antidepressants or nonsteroidal anti-inflammatories (NSAIDs). However, when these drugs are ineffective or not possible to use due to allergic reactions, other drug combinations may be prescribed. An effective medication for treatment of headaches is the combination of butalbital, acetaminophen and caffeine in the capsule form. McCrory et al., 41 HEADACHE 953-967 (2001). Although the role of each compound in the relief of headaches is incompletely understood, the combination of these three compounds due to their synergistic effects has been widely accepted as a treatment for various forms of headache.

Butalbital is a short to intermediate-acting barbiturate that acts as a depressant of the central nervous system. It is used in combination with other drugs for its sedative and relaxant effects in the treatment of headaches.

Caffeine is used in combination with other drugs for the treatment of headaches because of its role as an analgesic adjunct. Caffeine on its own does not have any known analgesic activity.

Acetaminophen, also known as paracetamol and sold under the trade name Tylenol®, is a clinically proven analgesic/antipyretic. Acetaminophen produces analgesia by elevation of the pain threshold and antipyresis through action on the hypothalamic heat-regulating center.

Delivery systems of over the counter or prescription drugs come in a variety of shapes and dosage forms, such as in a capsule, tablet, caplet, gel caplet (gelcap) or in a syrup form if a pill is impractical. However, due to consumer perception and patient preference, certain dosage forms have particular advantages over others. Until the 1980's, capsules were the preferred form for the delivery of active drugs. Capsules are a dosage form in which the drug is housed within two halves of gelatin shells that are banded together. Due to the ability of taking apart the capsule gelatin shell halves and then resealing the halves, tampering of the capsule became a major problem. For this reason, the pharmaceutical industry withdrew many of their capsule products from the market, often replacing them with tablets or caplets.

Tablets and caplets are compressed solid pills where the dosage forms are cylindrical or the oblong shape similar to capsules. Although both dosage forms are presently used in the market, tablets and caplets have not reached the same level of consumer acceptance that capsules once had. Consumer surveys suggest that a dosage form with the like appearance of a gelatin coated capsule is easier to swallow and that the drug contained in the capsule form will be more effective.

The gelcap is a drug product that may itself encompass many forms. For example, the gelcap may contain a filler containing the active drug in a liquid form that is encapsulated in a gelatin capsule like shell. Also, a gelcap can contain the active drug in a solid form, which has the shape of a caplet and a gelatin coating or covering to create the appearance and therefore, the consumer acceptability of the capsule.

There are numerous advantages to a drug being administered in the form of a solid gelcap. The gelcap can be manufactured in a wide variety of sizes, shapes and colors that consumers tend to find aesthetically appealing. Moreover, it has also been established that patient compliance is improved if a gelcap is used to administer the drug because of its soft and elastic nature, which makes it easier to swallow compared to a hard tablet or caplet.

A liquid gelcap also has numerous advantages. First, it retains many of the advantages of consumer acceptance and is easier to swallow due to the outer coating being a soft and elastic gelatin shell. Also, concentrated liquid compositions are well suited for encapsulation within a soft gelatin shell, creating flexibility that further assists in the capsule being easier to swallow. The active drug contained in the liquid form also provides advantages by dispersing the drug to the active site. For example, the active drug does not first have to dissolve in the gastrointestinal tract, thereby facilitating absorption of the pharmacologically active substance. See, for example, U.S. Pat. No. 6,689,382, which is expressly incorporated by reference herein. Other formulations take advantage of the liquid form by creating a sustained release gelatin capsule, thereby permitting the delivery of the drug in a controlled fashion. See, for example, U.S. Pat. Nos. 5,324,280 and 6,929,803, which are expressly incorporated by reference herein.

### SUMMARY OF THE INVENTION

The present invention provides compositions and methods of using the compositions for the therapeutic treatment of pain. Specifically, the present invention comprises a composition of a barbiturate, an analgesic/antipyretic and an analgesic adjunct in the form of a gelcap.

In another embodiment of the present invention, the compositions may comprise a barbiturate from one or more of the group consisting of amobarbital, butalbital aprobarbital, butabarbital, butethal, cyclobarbital, mephobarbital, methohexital, methylphenobarbital, pentobarbital, phenobarbital, secobarbital, talbutal, thiobarbital, thiamylal, thiopental and thiopental sodium.

In another embodiment of the present invention, the compositions may comprise an analgesic/antipyretic from one or more of the group consisting of acetaminophen, buprenorphine, butorphanol, codeine, dextropropoxyphene, dihydrocodeine, fentanyl, diamorphine (Heroin), hydrocodone, hydromorphone, ketobemidone, morphine, nalbuphine, oxycodone, oxymorphone, pentazocine, pethidine, tramadol, acetylsalicylic acid, diflunisal, ethenzamide, aminophenazone, metamizole, phenazone, phenacetin, ziconotide, tetrahydrocannabinol, acetylsalicylic acid (aspirin), choline salicylate magnesium salicylate, sodium salicylate, ibuprofen, naproxen and ketoprofen.

In another embodiment of the present invention, the compositions may comprise an analgesic adjunct from one or more of the group consisting of S (+)-ketamine, metoclopramide, ciramadol, sulfentanil, caffeine and remifentanil.

In one embodiment of the present invention, the compositions may comprise butalbital, acetaminophen and caffeine.

The compositions of the present invention may be administered to the patient for oral use and may be in the form of an elixir, syrup and/or suspension according to an individual patient's preferences. In another embodiment of the present invention, the compositions may further comprise a flavorant.

In another embodiment of the present invention, the compositions may be substantially free of other added active ingredients. The other added active ingredient may comprise another barbiturate, such as one or more of the group consisting of amobarbital, aprobarbital, butabarbital, butethal, cyclobarbital, mephobarbital, methohexital, methylphenobarbital, pentobarbital, phenobarbital, secobarbital, talbutal, thiobarbital, thiamylal, thiopental and thiopental sodium. The other added active ingredient may comprise another analgesic such as one or more of the group consisting of buprenorphine, butorphanol, codeine, dextropropoxyphene, dihydrocodeine, fentanyl, diamorphine (Heroin), hydrocodone, hydromorphone, ketobemidone, morphine, nalbuphine, oxycodone, oxymorphone, pentazocine, pethidine, tramadol, acetylsalicylic acid, diflunisal, ethenzamide, aminophenazone, metamizole, phenazone, phenacetin, ziconotide and tetrahydrocannabinol. The other added active ingredient may comprise another antipyretic such as one or more of the group consisting of acetylsalicylic acid (aspirin), choline salicylate magnesium salicylate, sodium salicylate, ibuprofen, naproxen and ketoprofen. The other added active ingredient may comprise another analgesic adjunct such as one or more of the group consisting of S (+)-ketamine, metoclopramide, ciramadol, sulfentanil and remifentanil.

In another embodiment, the compositions of the present invention may comprise one or more of about 25 mg to about 75 mg of butalbital; about 200 mg to about 600 mg of acetaminophen; and about 20 mg to about 60 mg of caffeine.

In another embodiment, the compositions of the present invention may comprise one or more of about 37.5 mg to about 62.5 mg of butalbital; about 300 mg to about 500 mg of acetaminophen; and about 30 mg to about 50 mg of caffeine.

In another embodiment, the compositions of the present invention may comprise one or more of about 45 mg to about 55 mg of butalbital; about 360 mg to about 440 mg of acetaminophen; and about 36 mg to about 44 mg of caffeine.

In another embodiment, the compositions of the present invention may comprise one or more of about 50 mg of butalbital, about 400 mg of acetaminophen, and about 40 mg of caffeine.

In another embodiment of the present invention, the compositions may be administered to a patient to treat and/or alleviate the occurrence or negative effects from one or more of the group consisting of chronic pain and acute pain.

In another embodiment of the present invention, the compositions may be administered to a patient to treat and/or alleviate the occurrence or negative effects of headaches.

In another embodiment, the compositions may be administered to a patient to treat and/or alleviate the occurrence or negative effects from one or more of the group consisting of tension type headache, migraine headache, cluster headache or chronic daily headache.

In another embodiment, the compositions may be administered to a patient to treat and/or alleviate the occurrence or negative effects from one or more of the group consisting of episodic tension type headache or chronic tension type headache.

The present invention also includes methods of administering the compositions of the invention to a patient to treat and/or alleviate the occurrence or negative effects of pain. Specifically, the methods may include administering to a patient a composition comprising a barbiturate, an analgesic/antipyretic and analgesic adjunct in the form of a gelcap.

In another embodiment, the methods may include the patient taking the compositions of the invention.

In another embodiment of the present invention, the methods may utilize a barbiturate selected from one or more of the group consisting of amobarbital, butalbital aprobarbital, butabarbital, butethal, cyclobarbital, mephobarbital, methohexital, methylphenobarbital, pentobarbital, phenobarbital, secobarbital, talbutal, thiobarbital, thiamylal, thiopental and thiopental sodium.

In another embodiment of the present invention, the methods may utilize an analgesic/antipyretic selected from one or more of the group consisting of acetaminophen, buprenorphine, butorphanol, codeine, dextropropoxyphene, dihydrocodeine, fentanyl, diamorphine (Heroin), hydrocodone, hydromorphone, ketobemidone, morphine, nalbuphine, oxycodone, oxymorphone, pentazocine, pethidine, tramadol, acetylsalicylic acid, diflunisal, ethenzamide, aminophenazone, metamizole, phenazone, phenacetin, ziconotide, tetrahydrocannabinol, acetylsalicylic acid (aspirin), choline salicylate magnesium salicylate, sodium salicylate, ibuprofen, naproxen and ketoprofen.

In another embodiment of the present invention, the methods may utilize an analgesic adjunct selected from one or more of the group consisting of S (+)-ketamine, metoclopramide, ciramadol, sulfentanil, caffeine and remifentanil.

In one embodiment of the present invention, the methods may include administering a composition comprising butalbital, acetaminophen and caffeine in the form of a gelcap.

In one embodiment of the present invention, the methods may include the patient taking a composition comprising butalbital, acetaminophen and caffeine in the form of a gelcap.

In another embodiment, the methods may include administering the compositions of the present invention to the patient orally.

In another embodiment of the present invention, the methods may utilize compositions in the form of an elixir, syrup and/or suspension according to an individual patient's preferences. In another embodiment of the present invention, the methods may utilize compositions comprising a flavorant.

In another embodiment of the present invention, the methods may utilize compositions substantially free of other added active ingredients. The other added active ingredient may be one or more of another barbiturate, such as amobarbital, aprobarbital, butabarbital, butethal, cyclobarbital, mephobarbital, methohexital, methylphenobarbital, pentobarbital, phenobarbital, secobarbital, talbutal, thiobarbital, thiamylal, thiopental and thiopental sodium. The other added active ingredient may be one or more of another analgesic such as buprenorphine, butorphanol, codeine, dextropropoxyphene, dihydrocodeine, fentanyl, diamorphine (Heroin), hydrocodone, hydromorphone, ketobemidone, morphine, nalbuphine, oxycodone, oxymorphone, pentazocine, pethidine, tramadol, acetylsalicylic acid, diflunisal, ethenzamide, aminophenazone, metamizole, phenazone, phenacetin, ziconotide and tetrahydrocannabinol. The other added active ingredient may be one or more of another antipyretic such as acetylsalicylic acid, choline salicylate magnesium salicylate, sodium salicylate, ibuprofen, naproxen and ketoprofen. The other added active ingredient may be one or more of another analgesic adjunct such as S (+)-ketamine, metoclopramide, ciramadol, sulfentanil and remifentanil.

In another embodiment, the methods may utilize compositions comprising one or more of about 25 mg to about 75 mg of butalbital; about 200 mg to about 600 mg of acetaminophen; and about 20 mg to about 60 mg of caffeine.

In another embodiment, the methods may utilize compositions comprising one or more of about 37.5 mg to about 62.5 mg of butalbital; about 300 mg to about 500 mg of acetaminophen; and about 30 mg to about 50 mg of caffeine.

In another embodiment, the methods may utilize compositions comprising one or more of about 45 mg to about 55 mg of butalbital; about 360 mg to about 440 mg of acetaminophen; and about 36 mg to about 44 mg of caffeine.

In another embodiment, the methods may utilize compositions comprising one or more of about 50 mg of butalbital; about 400 mg of acetaminophen; and about 40 mg of caffeine.

In another embodiment, the methods may include administering the compositions of the present invention to a patient to treat and/or alleviate the occurrence or negative effects from one or more of the group consisting of chronic pain or acute pain.

In another embodiment, the methods may include administering the compositions of the present invention to a patient to treat and/or alleviate the occurrence or negative effects of headaches.

In another embodiment, the methods may include administering the compositions of the present invention to a patient to treat and/or alleviate the occurrence or negative effects from one or more of the group consisting of tension type headache, migraine headache, cluster headache or chronic daily headache.

In another embodiment, the methods may include administering the compositions of the present invention to a patient to treat and/or alleviate the occurrence or negative effects from one or more of the group consisting of episodic tension type headache or chronic tension type headache.

In another embodiment of the present invention, the methods may utilize compositions that are administered to a patient at a frequency of once a day, twice a day, three times a day, four times a day, five times a day, six times a day, seven times a day, eight times a day, nine times a day, ten times a day, eleven times a day and twelve times a day.

In another embodiment of the present invention, the methods may utilize compositions that a patient takes at a frequency of once a day, twice a day, three times a day, four times a day, five times a day, six times a day, seven times a day, eight times a day, nine times a day, ten times a day, eleven times a day and twelve times a day.

In another embodiment of the present invention, the methods may utilize compositions that are administered to a patient at a frequency of once every hour, once every two hours, once every three hours, once every four hours, once every five hours, once every six hours, once every seven hours, once every eight hours, once every nine hours, once every ten hours, once every eleven hours, once every twelve hours, once every thirteen hours, once every fourteen hours, once every fifteen hours, once every sixteen hours, once every seventeen hours, once every eighteen hours, once every nineteen hours, once every twenty hours, once every twenty one hours, once every twenty two hours, once every twenty three hours and once every twenty four hours.

In another embodiment of the present invention, the methods may utilize compositions that a patient takes at a frequency of once every hour, once every two hours, once every three hours, once every four hours, once every five hours, once every six hours, once every seven hours, once every eight hours, once every nine hours, once every ten hours, once every eleven hours, once every twelve hours, once every thirteen hours, once every fourteen hours, once every fifteen hours, once every sixteen hours, once every seventeen hours, once every eighteen hours, once every nineteen hours, once every twenty hours, once every twenty one hours, once every twenty two hours, once every twenty three hours and once every twenty four hours.

In another embodiment, the methods may utilize compositions that are administered in a dose unit of 0.5, 1, 1.5, 1.5, 2, 2.5, 3, 3.5 and 4 dosage forms.

### DETAILED DESCRIPTION OF THE INVENTION

It is understood that the present invention is not limited to the particular methodologies, protocols, fillers, excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a barbiturate” is a reference to one or more barbiturates and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “patient,” as used herein, comprises any and all organisms and includes the term “subject.” “Patient” may refer to a human or any other animal, including mammals.

The term “administrable” defines a composition that is able to be given to a patient. Likewise, “administering” refers to the act of giving a composition to a patient or otherwise making such composition available to a patient or the patient taking a composition themselves.

The term “active ingredient” as used herein is any ingredient that is a barbiturate, an analgesic/antipyretic, and an analgesic adjunct when taken orally by a patient.

As used herein, the terms “inactive,” “inert,” “excipient,” and/or “formulatory” refer to any compound that is an inactive ingredient of a described composition. The definition of “inactive ingredient” as used herein follows that of the U.S. Food and Drug Administration, as defined in 21 C.F.R. 201.3(b)(8), which is any component of a drug product other than the active ingredient. By “active ingredient,” then, is meant any compound intended to furnish pharmacological activity or other direct effect in the diagnosis, cure, mitigation, treatment and/or prevention of a condition. See 21 C.F.R. 210.3(b)(7). Further, “active ingredients” include those compounds of the composition that may undergo chemical change during the manufacture of the composition and be present in the final composition in a modified form intended to furnish an activity or effect. Id.

The term “substantially free,” as used herein, means free from therapeutically effective amounts of compounds when administered in suggested dosages, but may include trace amounts of compounds in non-therapeutically effective amounts.

The term “dosage form,” as used herein, is the form in which the dose is to be administered to the patient. The drug is generally administered as part of a formulation that includes nonmedical agents, referred to as pharmaceutical ingredients. The dosage form has unique physical and pharmaceutical characteristics. Dosage forms may be solid, liquid or gaseous. Solid forms include, but are not limited to capsules, tablets, caplets, gel caplets (gelcap), lozenges, wafers etc. Liquid dosage forms include, but are not limited to, syrups, elixirs, injectable solutions, and intravenous solutions. Gaseous forms include vapors, inhalants, and the like.

As used herein the term “tablet” refers to a medication, usually mixed with a binder powder, which is molded and pressed into the form of a tablet, traditionally circular or disk-shaped. As used herein, the term “caplet” refers to a smooth, coated, oval-shaped tablet. As used herein, the term “capsule” refers to a solid dosage form in which the drug, in discrete units, is enclosed in a hard or soft soluble container, usually of a form of gelatin. The discrete units of the capsule dosage form include, but are not limited to, beads, granules, pellets, spheroids, particles, tablets, pills, etc. As used herein, the term “gelcap,” otherwise known as “gel caplet,” refers to a capsule-shaped dosage form where the active ingredients are dissolved in a liquid that is coated within a gelatin shell for easy swallowing or to have the appearance of easy swallowing.

Despite many advances in treatment, pain continues to be a major healthcare problem in the United States. Dorsey et al., 16 AACN CLIN. ISSUES 277-90 (2005). Pain is a condition that affects millions of Americans every day, costing in excess of $100 billion for treatment-related costs and lost work productivity. Id. Pain is an unpleasant sensation with a wide range in severity that can be localized or prevalent throughout the body. The Merck Manual of Medical Information (M. Beers et al., 2^{nd} Home ed., Merck Research Laboratories, 2003). Pain is affected by nerve stimulation that carries impulses to the brain and is a symptom of an underlying disease, disorder, or physical injury. Id.

Pain can be classified into two groups: acute pain or chronic pain. Id. According to the National Centers for Health Statistics, more than 50 million Americans experience chronic pain. NATIONAL CTR. FOR HEALTH STATISTICS, HEALTH, UNITED STATES, 2006 WITH CHARTBOOK ON TRENDS IN THE HEALTH OF AMERICANS 68-71 (2006). Chronic pain is a persisting or reoccurring pain that can last for months and even years. The Merck Manual of Medical Information (M. Beers et al, 2^{nd} Home ed., Merck Research Laboratories, 2003). It is more common for chronic pain to be due to a disorder such as sickle cell anemia or a serious physical injury.

Alternatively, acute pain tends to come on quickly, but subsides after a short time. Acute pain serves a useful purpose by alerting humans of a physiological hazard or a need for treatment. Therefore, acute pain tends to be caused from a sudden physical injury or infection.

Clinical complaints due to pain come in many varieties. Such complaints may be due to such ailments as arthritis, back pain, neuropathy, or a headache. Hence, the therapeutic treatment can be highly dependent on the type of pain. Such treatments may include acupuncture, change in nutrition, exercise, or medication.

One of the most common sources of pain are headaches. Headache symptoms may be due to tension type headache (tension headache), migraine, cluster headache or chronic daily headache. However, tension type headache is by far the most common form. Nearly 70% of all headaches are attributed to tension type headache and approximately 3% of population suffers from them. Olesen et al., 44 J. CLIN. EPIDEMIOL. 1147-57 (1991).

Tension headache is classified into two forms: episodic and chronic. Lipton et al., 279 JAMA 381-3 (1998). These forms distinguish between occasional headaches separated by varying lengths of time between attacks and frequent headaches that occur, in many cases, almost daily. Id.

Episodic tension headache occurs on fewer than 15 days a month. Juang et al., 66 NEUROLOGY 160-1 (2006). These headaches are usually brief, lasting a few minutes to a few hours. In one survey of people with episodic tension headache, over 60% had scalp and neck muscle tenderness in addition to head pain. People with increasingly frequent attacks of the episodic form may be at higher risk of developing the chronic form of the headache over a period of years.

Chronic tension headache occurs on 15 days a month or more for at least three months. Id. Compared with the episodic form, chronic tension headache is less common, but twice as many women as men have the chronic form. The duration and the severity of episodic and chronic tension headaches are similar, although for many people with the chronic form, pain is daily and almost continuous. Like the episodic form, chronic tension headache can be with or without scalp tenderness.

There has been extensive clinical trials that have tested the collaborative effect of various combinations of drugs towards episodic and chronic tension type headache. Diserio et al., 10 CLIN. THER. 69-81 (1987). For example, clinical trials have researched the combination of a barbiturate, an analgesic/antipyretic and an analgesic adjunct such as butalbital, acetaminophen and caffeine. Id.

In another embodiment, the compositions and methods of the present invention may comprise a barbiturate comprising one or more of amobarbital, butalbital, aprobarbital, butabarbital, butethal, cyclobarbital, mephobarbital, methohexital, methylphenobarbital, pentobarbital, phenobarbital, secobarbital, talbutal, thiobarbital, thiamylal, thiopental and thiopental sodium. For example, methohexital binds to the GABA A receptor and therefore has anesthetic properties that may be beneficial in the treatment of various forms of headache. Wennberg et al., 286 J. PHARMACOL. EXP. THER. 1177-82 (1998).

Butalbital is a short to intermediate acting central nervous system depressant and is a derivative of barbituric acid. Butalbital (5-allyl-5-isobutylbarbituric acid) is a white odorless powder that is slightly soluble in cold water. Medications containing butalbital have been extensively studied in clinical trials for treatment of tension type headache. McCrory et al., 41 HEADACHE 953-67 (2001). Butalbital acts as an allosteric modulator by binding at a site associated with the GABA A receptor. The GABA A receptor is a pentameric transmembrane chloride ion channel that binds GABA. Because of the binding to butalbital, the duration time is increased for which the Cl⁻ ionophore on the GABA A receptor is open. This opening allows chloride ions to enter the postjunctional terminal which results in inhibition of the postsynaptic neuron. Goadsby et al., 134 B. J. PHARMACOL. 896-904 (2001). Inhibition of the postsynaptic neuron therefore explains butalbital's central nervous system depressant effects.

In one specific embodiment, the compositions and methods of the present invention may comprise butalbital. Specifically, the amounts may range from about 25 mg to about 75 mg. In another specific embodiment, the compositions and methods of the present invention may comprise butalbital in amounts ranging from about 37.5 mg to about 62.5 mg. In another specific embodiment, the compositions and methods of the present invention may comprise butalbital in amounts ranging from about 45 mg to about 55 mg. In another specific embodiment, the compositions and methods of the present invention may comprise butalbital in an amount of about 50 mg.

In another embodiment, the compositions and methods of the present invention may comprise an analgesic/antipyretic comprising one or more of acetaminophen, buprenorphine, butorphanol, codeine, dextropropoxyphene, dihydrocodeine, fentanyl, diamorphine (Heroin), hydrocodone, hydromorphone, ketobemidone, morphine, nalbuphine, oxycodone, oxymorphone, pentazocine, pethidine, tramadol, acetylsalicylic acid, diflunisal, ethenzamide, aminophenazone, metamizole, phenazone, phenacetin, ziconotide, tetrahydrocannabinol, acetylsalicylic acid (aspirin), choline salicylate magnesium salicylate, sodium salicylate, ibuprofen or other non steroidal anti-inflammatory drugs (NSAIDs), naproxen and ketoprofen. For example, ibuprofen inhibits the role of COX-2 to induce prostaglandin synthesis. Although the inhibition of COX-2 primarily inhibits the inflammatory response, ibuprofen and other NSAIDs are effective analgesics and antipyretics.

Acetaminophen is an analgesic/antipyretic that is commonly used to reduce fever and headaches. Acetaminophen is also used in a combination with other active drugs in managing severe pain. The chemical name is N-(4-hydroxyphenyl)acetamide. Research suggests that acetominophen's analgesic/antipyretic activity is due to its ability to inhibit prostaglandin synthesis. Prostaglandin synthesis, a pathway that is directly linked to the symptoms of pain and fever, is induced by Cyclooxygenase isozymes (COX enzymes). Clottes et al., 71 MOL. PHARMACOL. 407-15 (2007); Botting, 31 CLIN. INFECT. DIS. S202-10 (2000). The discovery and research on various isoforms of COX enzymes, such as COX-3, demonstrate a strong inhibition by acetaminophen. Simmons et al., 99 PROC. NATL. ACAD. SCI. 13371-73 (2002). The role of various COX isozymes, and their effect on prostaglandin synthesis, is currently the prevailing theory on how acetaminophen reduces pain and fever.

In another specific embodiment, the compositions and methods of the present invention may comprise acetaminophen. Specifically, the amounts may range from about 200 mg to about 600 mg. In another specific embodiment, the compositions and methods of the present invention may comprise acetaminophen in amounts ranging from about 300 mg to about 500 mg. In another specific embodiment, the compositions and methods of the present invention may comprise acetaminophen in amounts ranging from about 360 mg to about 440 mg. In another specific embodiment, the compositions and methods of the present invention may comprise acetaminophen in an amount of about 400 mg.

In another embodiment, the compositions and methods of the present invention may comprise an analgesic adjunct comprising one or more of caffeine, S (+)-ketamine, metoclopramide, ciramadol, remifentanil and an opioid analgesic such as sulfentanil. For example, sulfentanil is effective in aiding pain relief where pain is severe and the relief is only required for a short period of time. Nahata et al., 10 CLIN. PHARM. 581-93 (1991). Therefore, sulfentanil may be effective as an adjunct for cluster headache, which is known for its severity and for symptoms that usually do not last longer than 30 minutes.

Caffeine is most commonly used as a central nervous stimulant due to its effect of warding off drowsiness. The chemical name of caffeine is 1,3,7-trimethyl-1H-purine-2,6(3H,7H)-dione. Caffeine is also used in combination for the treatment of headaches because of its role as an analgesic adjunct. Caffeine on its own does not have any known analgesic activity. When caffeine is combined with an analgesic such as acetaminophen, caffeine's analgesic adjunct activity provides a synergistic effect that enhances pain relief and provides the option of lowering the dosage of the analgesic. Engelhardt et al., 39 NEUROPHARMACOLOGY 2205-13 (2000). Research indicates that caffeine's role as an analgesic adjunct is due to its ability to induce the constriction of cerebral blood vessels, which leads to a decrease in cerebral blood flow and in the oxygen tension of the brain.

In another specific embodiment, the compositions and methods of the present invention may comprise caffeine. Specifically, the amounts may range from about 20 mg to about 60 mg. In another specific embodiment, the compositions and methods of the present invention may comprise caffeine in amounts ranging from about 30 mg to about 50 mg. In another specific embodiment, the compositions and methods of the present invention may comprise caffeine in amounts ranging from about 36 mg to about 44 mg. In another specific embodiment, the compositions and methods of the present invention may comprise caffeine in an amount of about 40 mg.

In another specific embodiment, the methods and compositions of the present invention may be free of other added active ingredients. The addition of other active ingredients can produce adverse side effects that can inhibit or outweigh the benefits of the methods and compositions of the present invention. For example, codeine is an opiate that can be used for its analgesic and antitussive properties. However, moderate use of codeine has been associated with a high number of gastrointestinal adverse effects including nausea, vomiting and abdominal pains. McCrory et al., 41 HEADACHE 953-67 (2001). In a specific embodiment, the compositions and methods of the present invention may be free of added codeine.

Secobarbital and pentobarbital are short acting barbiturates that when administered to patients in various doses, result in severe withdrawal syndrome. Id. Such symptoms related to withdrawal include delirium, seizures and hallucinations. Id. In a specific embodiment, one or more of the compositions and methods of the present invention may be free of added secobarbital and pentobarbital.

Non-steroidal anti-inflammatory drugs (NSAIDs) such as ibuprofen or aspirin are widely used for their analgesic and anti-inflammatory effects. However, current evidence suggests that adverse side effects, such as gastrointestinal and other bleeding risks with NSAIDs, probably outweigh its potential benefits. Kim et al., 58 J. PHARM. PHARMACOL. 1295-1304 (2006). In a specific embodiment, the compositions and methods of the present invention may be free of added non-steroidal anti-inflammatory drugs such as ibuprofen or aspirin.

Pemoline is a central nervous system stimulant that was commonly used to treat attention-deficit hyperactivity disorder (ADHD). Although pemoline is a stimulant that has various benefits such as in treating learning disorders, excessive intake of the drug has been linked to severe hepatotoxicity. Gardner et al., 40 J. AM. ACAD. CHILD. ADOLESC. PSYCHIATRY 622-29 (2001). In a specific embodiment, the compositions and methods of the present invention may be free of added pemoline.

Remifentanil is an analgesic that is commonly administered to patients as an analgesic adjunct after surgery. Moreover, remifentanil has strong sedative effects and therefore is also useful as a general anesthetic. However, the sedative effects are linked to severe dizziness shortly after administering the drug, which has presently limited the drug's use to surgery related anesthesia. Verbrugge et al., 101 ANESTH. ANALG. 365-70 (2005). In a specific embodiment, the compositions and methods of the present invention may be free of added remifentanil.

The composition and methods of the present invention provide the patient with the option of various dosage forms for the prevention or treatment of headaches.

In another specific embodiment of the present invention, the compositions may have a dosage form shape comprising no sharp edges and a smooth and uniform surface. Among other dosage forms apparent to the skilled artisan, the solid oral dosage form may be a tablet, a discrete unit-filled capsule, a gelcap or a caplet.

In another specific embodiment, the compositions of the present invention may be a solid dosage form which has a coating or gelatin covering and therefore, the appearance of the capsule, and are often referred to as a solid gelcap. Such gelcaps may consist of a solid caplet or tablet that is covered in opposite ends in a soft gelatin shell of different colors to simulate a capsule-like dosage form. See, for example, U.S. Pat. Nos. 5,824,338, 5,415,868, 5,317,849, 5,089,270, 4,966,771 and 4,820,524, which are expressly incorporated by reference herein.

In another specific embodiment, the compositions of the present invention may be in the form of a liquid gelcap which may consist of a filler comprising one or more pharmaceutically active materials dissolved or dispersed in an appropriate liquid vehicle encapsulated in a gelatin shell generally comprising gelatin together with a plasticizer such as glycerin or sorbitol. The filler material may comprise, for example, polyethylene glycols. See, for example, U.S. Pat. Nos. 4,780,316; 5,419,916; 5,641,512; and 6,589,536 which are expressly incorporated by reference herein.

A liquid gelcap has numerous advantages. First, it retains many of the advantages of consumer acceptance and is easier to swallow due to the outer coating being a soft and elastic gelatin shell. Also, concentrated liquid compositions are well suited for encapsulation within a soft gelatin shell, creating flexibility that further assists in the capsule being easier to swallow. The active drug contained in the liquid form also has advantages in dispersing the drug to the active site. For example, the active drug does not first have to dissolve in the gastrointestinal tract, thereby facilitating absorption of the pharmacologically active substance. See, for example, U.S. Pat. No. 6,689,382 which is expressly incorporated by reference herein. Other formulations take advantage of the liquid form by creating a sustained release gelatin capsule, thereby permitting the delivery of the drug in a controlled fashion. See, for example, U.S. Pat. Nos. 5,324,280 and 6,929,803, which are expressly incorporated by reference herein. Many shell and fill formulations are discussed in “Advances in Softgel Formulation Technology”, M. S. Patel, F. S. S. Morton and H. Seager, Manufacturing Chemists, July 1989; “Soft Elastic Gelatin Capsules: A Unique Dosage Form”, William R. Ebert, Pharmaceutical Technology, October 1977; and “Soft gelatin capsules: a solution to many tableting problems”, H. Seager, Pharmaceutical Technology, September 1985.

To prepare the compositions of the present invention, each of the active ingredients may be combined in intimate admixture with a suitable carrier according to conventional compounding techniques. In a specific embodiment of the compositions of the present invention, the surface of the compositions may be coated with a polymeric film. Such a film coating has several beneficial effects. First, it reduces the adhesion of the compositions to the inner surface of the mouth, thereby increasing the patient's ability to swallow the compositions. Second, the film may aid in masking the unpleasant taste of certain drugs. Third, the film coating may protect the compositions of the present invention from atmospheric degradation. Polymeric films that may be used in preparing the compositions of the present invention include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia. Pharmaceutical carriers and formulations for swallowable compounds are well known to those of ordinary skill in the art. See generally, Handbook of Pharmaceutical Excipients (2nd ed. Wade and Waller eds., 1994).

In addition to those described above, any appropriate fillers and excipients may be utilized in preparing the compositions of the present invention so long as they are consistent with the objectives described herein. For example, binders are substances used to cause adhesion of powder particles in granulations. Such compounds appropriate for use in the present invention include, by way of example and without limitation, acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium, and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, polyethylene glycol, and others known to those of ordinary skill in the art.

Diluents also may be included in the compositions of the present invention in order to enhance the granulation of the compositions. Diluents can include, by way of example and without limitation, microcrystalline cellulose, sucrose, dicalcium phosphate, starches, and polyols of less than 13 carbon atoms, such as mannitol, xylitol, sorbitol, maltitol, and pharmaceutically acceptable amino acids, such as glycin, and their mixtures.

Lubricants are substances used in composition formulations that reduce friction during composition compression. Lubricants that may be used in the present invention include, by way of example and without limitation, stearic acid, calcium stearate, magnesium stearate, zinc stearate, talc, mineral and vegetable oils, benzoic acid, poly-(ethylene glycol), glyceryl behenate, stearyl fumarate, and others known to those of ordinary skill in the art.

Glidants improve the flow of powder blends during manufacturing and minimize composition weight variation. Glidants that may be used in the present invention include by way of example and without limitation, silicon dioxide, colloidal or fumed silica, magnesium stearate, calcium stearate, stearic acid, cornstarch, talc and others known to those of ordinary skill in the art.

If desired, compositions may be sugar coated or enteric coated by standard techniques.

The compositions of the present invention may be prepared using conventional methods and materials known in the pharmaceutical art. For example, U.S. Pat. Nos. 5,215,754 and 4,374,082 relate to methods for preparing such compositions, which are expressly incorporated by reference herein. Further, all pharmaceutical carriers and formulations described herein are well known to those of ordinary skill in the art, and determination of workable proportions in any particular instance will generally be within the capability of the person skilled in the art. Details concerning any of the excipients of the invention may be found in WADE & WALLER, supra. All active ingredients, fillers and excipients are commercially available from companies such as Sigma-Aldrich, St. Louis, Mo.; FMC Corp, Philadelphia, Pa.; Bayer, Germany; BASF, Germany; Mallinckrodt, Hazelwood, Mo.; Rhodia, France; ISP, Wayne, N.J.; and others.

A specific embodiment of the present invention may comprise compositions packaged in blister packs. Blister packs as packaging for compositions are well known to those of ordinary skill in the art. Blister packs may be made of a transparent plastic sheet which as been formed to carry a matrix of depression or blisters. One or more compositions are received in each depression or blister. A foil or plastic backing is then adhered across the plane of the sheet sealing the compositions in their respective blisters. Examples of materials used for the blister packs include, but are not limited to, aluminum, paper, polyester, PVC, and polypropylene. Alternative materials are known to those of ordinary skill in the art. To remove a composition, the depression material is pressed in and the composition is pushed through the backing material. Multiple blister packs may be placed in an outer package, often a box or carton for sale and distribution.

Another specific embodiment of the present invention may comprise compositions packaged in bottles. The bottle may be glass or plastic in form with a pop or screw top cap. Bottle packaging for such compositions are well known to those of ordinary skill in the art.

Additionally, the unit dose forms may be individually wrapped, packaged as multiple units on paper strips or in vials of any size, without limitation. The compositions of the invention may be packaged in unit dose, rolls, bulk bottles, blister packs and combinations thereof, without limitation.

In other embodiments, the composition may be an elixir, syrup and/or suspension. As used herein, the term “syrup” refers to a concentrated, aqueous preparation of a sugar or sugar substitute with or without an added flavoring agent. As used herein, the term “elixir” refers to a clear, sweetened, hydroalcoholic solution intended for oral use, and may or may not have an added flavoring agent. As used herein, a “suspension” is a preparation containing finely divided drug particles distributed somewhat uniformly throughout a vehicle in which the drug exhibits a minimum degree of solubility. Although water itself may make up the entire carrier, typical formulations may contain a co-solvent, for example and without limitation, propylene glycol and/or glycerin, to assist solubilization and incorporation of water insoluble ingredients, flavorants and the like into the composition. Any such ingredients may be included as desired or needed within the compositions and methods of the present invention as long as they are consistent with the objectives herein defined. For example, it is contemplated that when desirable, flavoring, preserving, suspending, thickening and/or emulsifying agents may be included in the compositions and methods of the present invention. Formulations for orally administered medications are well known in the art. Descriptions of suitable formulations may be found in Remington, The Science and Practice of Pharmacy (A. Gennaro ed., 20^{th} ed., Lippincott, Williams & Wilkins, 2000).

Flavorants that may be used in accordance with the present invention include those known to those skilled in the art. These flavorants may include, for example and without limitation, natural, artificial and synthetic flavor oils and flavoring aromatic and/or oils, oleoresins and extracts derived from plants, animals, leaves, flowers, fruits, and so forth, and combinations thereof. Non-limiting representative flavor oils include anise oil, cinnamon oil, peppermint oil, spearmint oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil. Also useful flavorants include fruit essences including apple essence, pear essence, peach essence, berry essence, wildberry essence, date essence, blueberry essence, kiwi essence, strawberry essence, raspberry essence, cherry essence, plum essence, pineapple essence, and apricot essence. Other useful flavorants include aldehydes and esters such as benzaldehyde (cherry, almond), citral, i.e., alpha-citral (lemon, lime), neural, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenyl (citrus, mandarin), mixtures thereof and the like. Honey and artificial honey flavor, as well as natural mixed berry flavor, citric acid, malic acid, vanilla, vanillin, cocoa, chocolate, and menthol may also be used in accordance with the present invention. Flavorants appealing to non-human patients may also be included in the composition of the invention, including but not limited to, yeast extract, meat extract, fish extract, poultry extract, cheese and other dairy flavors, and the like.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

### EXAMPLES

Without requiring further elaboration, it is believed that one skilled in the art, using the preceding description, can utilize the present invention to the fullest extent. The following examples are illustrative only, and not limiting of the remainder of the disclosure in any way whatsoever.

### Example 1

A composition of the following formulation is prepared in gelcap form, including the appropriate excipients, by standard methods known to those of ordinary skill in the art:

| | |
|---|---|
| Butalbital | 50 mg |
| Acetaminophen | 400 mg |
| Caffeine | 40 mg |

### Example 2

A study is undertaken to evaluate the effectiveness of the compositions of the present invention in the treatment of patients. The objective of the study is to determine whether oral intake of the compositions and the combination of the compositions of the present invention results in a rapid improvement of the symptoms of tension type headache.

A double-blind, placebo controlled study is conducted over a 4 hour period. A total of 120 subjects, all presenting for treatment of symptoms of tension type headache, are chosen for the study. The patients range in age from 18 to 65 years old.

An initial assessment of the symptoms of each patient is conducted when the patients initially present for treatment. The patient rates the severity of the symptoms on a 4-point scale (0: absent; 1: mild; 2: moderate; 3: severe). For inclusion in the study, a patient must be rated with a score of two or above for tension type headache.

The 120 subjects chosen for the study are separated into four separate groups of 30. The characteristics of the symptoms between the four groups are comparable. The first group is administered a 2 gelcap dose of the composition of the present invention at the onset of the symptoms of tension type headache where the severity has reached at least a score of 2 as scored by the patient. The second group is administered a placebo medication at the onset of the symptoms of tension type headache that is similar in all respects to the administered composition except for the exclusion of the active ingredients butalbital, acetaminophen and caffeine. The third group is administered a placebo medication at the onset of the symptoms of tension type headache that is similar in all respects to the administered composition except for the exclusion of the active ingredient butalbital. The fourth group is administered a placebo medication at the onset of the symptoms of tension type headache that is similar in all respects to the administered composition except for the exclusion of the active ingredients butalbital and caffeine. The various symptoms of tension type headache are evaluated by the patient 0.5, 1, 2, 3 and 4 hours after ingestion of the study medication using the same 4-point scale. The symptoms evaluated are pain severity, tense and uptight feeling, and muscle stiffness.

The assessment of the relief for pain severity, tense and uptight feeling, and muscle stiffness is conducted for each subject group. The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 0.5, 1, 2, 3 and 4 hours, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

This study will demonstrate the efficacy of the composition of the present invention in treating the symptoms of tension type headache. Regarding potential adverse effects of taking the medication, if there are no significant differences between the four therapeutic groups, this study will demonstrate that the administration of the composition of the present invention is effective at treating symptoms of tension type headache, in addition to being well-tolerated by the patients.

### Example 3

A study is undertaken to evaluate the effectiveness of the compositions of the present invention in the treatment of patients. The objective of the study is to determine whether oral intake of the compositions and the combination of the compositions of the present invention results in a rapid improvement of the symptoms of joint pain due to rheumatoid arthritis

A double-blind, placebo controlled study is conducted over a 4 hour period. A total of 120 subjects, all presenting for treatment of symptoms of joint pain due to rheumatoid arthritis, are chosen for the study. The patients range in age from 18 to 65 years old.

An initial assessment of the symptoms of each patient is conducted when the patients initially present for treatment. The patient rates the severity of the symptoms on a 4-point scale (0: absent; 1: mild; 2: moderate; 3: severe). For inclusion in the study, a patient must be rated with a score of two or above for joint pain due to rheumatoid arthritis.

The 120 subjects chosen for the study are separated into four separate groups of 30. The characteristics of the symptoms between the four groups are comparable. The first group is administered a 2 gelcap dose of the composition of the present invention at the onset of the symptoms of joint pain due to rheumatoid arthritis where the severity has reached at least a score of 2 as scored by the patient. The second group is administered a placebo medication at the onset of the symptoms of joint pain due to rheumatoid arthritis that is similar in all respects to the administered composition except for the exclusion of the active ingredients butalbital, acetaminophen and caffeine. The third group is administered a placebo medication at the onset of the symptoms of joint pain due to rheumatoid arthritis that is similar in all respects to the administered composition except for the exclusion of the active ingredient butalbital. The fourth group is administered a placebo medication at the onset of the symptoms of joint pain due to rheumatoid arthritis that is similar in all respects to the administered composition except for the exclusion of the active ingredients butalbital and caffeine. The joint pain is evaluated in severity by the patient 0.5, 1, 2, 3 and 4 hours after ingestion of the study medication using the same 4-point scale.

The assessment of the relief for joint pain severity is conducted for each subject group. The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 0.5, 1, 2, 3 and 4 hours, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

This study will demonstrate the efficacy of the composition of the present invention in treating the symptoms of joint pain due to rheumatoid arthritis. Regarding potential adverse effects of taking the medication, if there are no significant differences between the four therapeutic groups, this study will demonstrate that the administration of the composition of the present invention is effective at treating symptoms of joint pain due to rheumatoid arthritis, in addition to being well-tolerated by the patients.

While specific embodiments of the present invention have been described, other and further modifications and changes may be made without departing from the spirit of the invention. All further and other modifications and changes are included that come within the scope of the invention as set forth in the claims. The disclosures of all publications cited above are expressly incorporated by reference in their entireties to the same extent as if each were incorporated by reference individually.

1. A composition comprising a barbiturate, an analgesic/antipyreffc and an analgesic adjunct, wherein said composition is in the fbrm of a gelcap and wherein said composition is free of other added active ingredients.

2. The composition of claim 1, wherein said barbiturate is selected from one or more of the group consisting of amobarbital, butalbital, aprobarbital, butabarbital, butethal, cyclobarbital. mephobarbital. methohexital, methylphenobarbitaL pentobarbital, phenobarbital, secobarbital, talbutal, thiobarbital, thiamylal. thiopental and thiopental sodium.

3. The composition of claim 1, wherein said analgesic/antipyretic is selected from one or more of the group consisting of acetaminophen, buprenorphine, butorphanol, codeine, dextropropoxyphene, dihydrocodeine, fentanyl, diamorphine (Heroin), hydrocodone, hydromorphone, ketobemidone, morphine, nalbuphine, oxycodone, oxymorphone. pentazocine, pethidine, tramadol, acetylsalicylic acid, diflunisal ethenzamide, aminophenazone, metamizole, phenazone, phenacetin, ziconotide, tetrahydrocannabinol, acetylsalicylic acid (aspirin), choline salicylate magnesium salicriate, sodium salicylate, ibuprofen, naproxen and ketoprofen.

4. The composition of claim 1, wherein said analgesic adjunct is selected from one or more of the group consisting of S (+)-ketamine, metoclopramide, ciramadol, sufentanil, caffeine and remifentanil.

5. The composition of claim 1 wherein said barbiturate is butalbital, said analgesic/antipyretic is acetaminophen and said analgesic adjunct is caffeine, and wherein said composition is administrable to a patient.

6. The composition of claim 5, wherein said composition is administrable to aid patient orally.

7. The composition of claim 1, wherein said composition comprises one or more of the group consisting of elixir, syrup and suspension.

8. The composition of claim 1, wherein said composition further comprises a flavorant.

9. (canceled)

10. (canceled)

11. (canceled)

12. (canceled)

13. (canceled)

14. (canceled)

15. (canceled)

16. The composition of claim 5, wherein said butalbital is present in the range of about 25 mg to about 75 mg.

17. The composition of claim 5, wherein said acetaminophen is present in the range of about 200 mg to about 600 mg.

18. The composition of claim 5, wherein said caffeine is present in the range of about 20 mg to about 60 mg.

19. The composition of claim 5, wherein said butalbital is present in the range of about 37.5 mg to about 62.5 mg.

20. The composition of claim 5, wherein said acetaminophen is present in the range of about 300 mg to about 500 mg.

21. The composition of claim 5, wherein said caffeine is present in the range of about 30 mg to about 50 mg.

22. The composition of claim 5, wherein said butalbital is present in the range of about 45 mg to about 55 mg.

23. The composition of claim 5, wherein said acetaminophen is present in the range of about 360 mg to about 440 mg.

24. The composition of claim 5, wherein said caffeine is present in the range of about 36 mg to about 44 mg.

25. The composition of claim 5, wherein said composition comprises about 25 mg to about 75 mg of butalbital; about 200 mg to about 600 mg of acetaminophen: and about 20 mg to about 60 mg of caffeine.

26. The composition of claim 5, wherein said composition comprises about 37.5 mg to about 62.5 mg of butalbital; about 300 mg to about 500 mg of acetaminophen; and about 30 mg to about 50 mg of caffeine.

27. The composition of claim 5, wherein said composition comprises about 45 mg to about 55 mg of butalbital: about 360 mg to about 440 mg of acetaminophen; and about 36 mg to about 44 mg of caffeine.

28. The composition of claim 27, wherein said butalbital is present in the amount of about 50 mg.

29. The composition of claim 27, wherein said acetaminophen is present in the amount of about 400 mg.

30. The composition of claim 27, wherein said caffeine is present in the amount of about 40 mg.

31. The composition of claim 5, wherein said butalbital is present in the amount of about 50 mg; said acetaminophen is present in the amount of about 400 mg: and said caffeine is present in the amount of about 40 mg.

32. The composition of claim 5, wherein said composition is administrable to said patient to treat and/or alleviate the occurrence or negative effects of pain.

33. The composition of claim 32, wherein said pain is from one or more of the group consisting of chronic pain or acute pain.

34. The composition of claim 33, wherein said composition is administered to said patient to treat and/or alleviate the occurrence or negative effects of headaches.

35. The composition of claim 34, wherein said headaches is from one or more of the group consisting of tension type headache, migraine headache, cluster headache or chronic daily headache.

36. The composition of claim 35, wherein said tension type headaches is from one or more of the group consisting of episodic or chronic.

37. A method comprising administering to a patient the composition of claim 1.

38. A method comprising a patient taking the composition of claim 1,

39. The method of claim 37, wherein said barbiturate is selected from one or more of the group consisting of amobarbital, butalbital, aprobarbital, butabarbital, butethal, cyclobarbital mephobarbital, methohexital, methylphenobarbital, pentobarbital, phenobarbital, secobarbital, talbutal, thiobarbital, thiamylal, thiopental and thiopental sodium.

40. The method of claim 37, wherein said analgesic/antipyretic is selected from one or more of the group consisting of acetaminophen, buprenorphine, butorphanol, codeine, dextropropoxyphene, dihydrocodeine, fentanyl, diamorphine (Heroin), hydrocodone, hydromorphone, ketobemidone, morphine, nalbuphine, oxycodone, oxymorphone, pentazocine, pethidine, tramadol, acetylsalicylic acid, diflunisal, ethenzamide, aminophenazone, metamizole, phenazone, phenacetin, ziconotide, tetrahydrocannabinol, acetylsalicylic acid (aspirin), choline salicylate magnesium salicylate, sodium salicylate, ibuprofen, naproxen and ketoprofen.

41. The method of claim 37, wherein said analgesic adjunct is selected from one or more of the group consisting of S (+)-ketamine. metoclopramide, ciramadol, sufentanil, caffeine and remifentanil.

42. A method comprising administering to a patient the composition of claim 5.

43. A method comprising a patient taking the composition of claim 5.

44. The method of claim 37, wherein said composition is administered to th patient orally.

45. The method of claim 37, wherein said composition comprises one or more of the group consisting of elixir, syrup and suspension.

46. The method of claim 37, wherein said composition further comprises a flavorant.

47. The method of claim 37, wherein said composition is substantially free of other added active ingredients.

48. The method of claim 47, wherein said other active ingredient is another barbiturate.

49. The method of claim 47, wherein said other active ingredient is another analgesic/antipyretic.

50. The method of claim 47, wherein said other active ingredient is another analgesic adjunct.

51. The method of claim 48, wherein said barbiturate is selected from one or more of the group consisting of amobarbital, aprobarbital, butabarbital, butethal, cyclobarbital, mephobarbital, methohexital, methylphenobarbital, pentobarbital, phenobarbital, secobarbital, talbutal, thiobarbital, thiamylal thiopental and thiopental sodium.

52. The method of claim 49, wherein said analgesic/antipyretic is selected from one or more of the group consisting of buprenorphine, butorphanol, codeine, dextropropoxyphene, dihydrocodeine, fentanyl, diamorphine (Heroin), hydrocodone, hydromorphone, ketobemidone morphine, nalbuphine, oxycodone, oxymorphone, pentazocine, pethidine, tramadol, acetylsalicylic acid, diflunisal, ethenzamide, aminophenazone, metamizole, phenazone, phenacetin, ziconotide, tetrahydrocannabinol, acetylsalicylic acid (aspirin), choline salicylate magnesium salicylate, sodium salicylate, ibuprofen, naproxen and ketoprofen.

53. The method of claim 50, wherein said analgesic adjunct is selected from one or more of the group consisting of S (+)-ketamine, metoelopramide, ciramadol, sufentanil and remifentanil.

54. The method of claim 42, wherein said butalbital is present in the range of about 25 mg to about 75 mg.

55. The method of claim 42, wherein said acetaminophen is present in the range of about 200 mg to about 600 mg.

56. The method of claim 42, wherein said caffeine is present in the range of about 20 mg to about 60 mg.

57. The method of claim 42, wherein said butalbital is present in the range of about 37.5 mg to about 62.5 mg.

58. The method of claim 42, wherein said acetaminophen is present in the range of about 300 mg to about 500 mg.

59. The method of claim 42, wherein said caffeine is present in the range of about 30 mg to about 50 mg.

60. The method of claim 42, wherein said butalbital is present in the range of about 45 mg to about 55 mg.

61. The method of claim 42, wherein said acetaminophen is present in the range of about 360 mg to about 440 mg.

62. The method of claim 42, wherein said caffeine is present in the range of about 36 mg to about 44 mg.

63. The method of claim 42, wherein said butalbital is about 50 mg.

64. The method of claim 42, wherein said acetaminophen is about 400 mg.

65. The method of claim 42, wherein said caffeine is about 40 mg.

66. The method of claim 42, wherein said composition is present in the range of about 25 mg to about 75 mg of said butalbital; about 200 mg to about 600 mg of said acetaminophen: and about 20 mg to about 60 mg of said caffeine.

67. The method of claim 42, wherein said composition is present in the range of about 37.5 mg to about 62.5 mg of said butalbital; about 300 mg to about 500 mg of said acetaminophen; and about 30 mg to about 50 mg of said caffeine.

68. The method of claim 42, wherein said composition is present in the range of about 45 mg to about 55 mg of said butalbital; about 360 mg to about 440 mg of said acetaminophen: and about 36 mg to about 44 mg of said caffeine.

69. The method of claim 42, wherein said butalbital is present in the amount of about 50 mg; said acetaminophen is present in the amount of about 400 mg: and s id caffeine is present in the amount of about 40 mg.

70. The method of claim 42, wherein said composition is administered to said patient to treat and/or alleviate the occurrence or negative effects of pain.

71. The method of claim 42, wherein said composition is administered to said patient to treat and/or alleviate the occurrence or negative effects from one or more of the group consisting of chronic pain or acute pain.

72. The method of claim 42, wherein said composition is administered to said patient to treat and/or alleviate the occurrence or negative effects of headaches.

73. The method of claim 42, wherein said composition is administered to said patient to treat and/or alleviate the occurrence or negative effects from one or more of the group consisting of tension type headache, migraine headache, cluster headache or chronic daily headache.

74. The method of claim 42, wherein said composition is administered to said patient to treat and/or alleviate the occurrence or negative effects from one or more of the group consisting of episodic tension type headache or chronic tension type headache.

75. The method of claim 42, wherein said composition is administered to said patient at a frequency selected from the group consisting of once a day, twice a day, three times a day, four times a day, five times a day, six times a day, seven times a day, eight times a day, nine times a day, ten times a day, eleven times a day and twelve times a day.

76. The method of claim 42, wherein said patient takes said composition at a frequency selected from the group consisting of once a day, twice a day, three times a day. four times a day, five times a day, six times a day, seven times a day, eight times a day, nine times a day, ten times a day, eleven times a day and twelve times a day.

77. The method of claim 42, wherein said composition is administered to said patient at a frequency selected from the group consisting of once every hour, once every two hours, once every three hours, once every four hours, once every five hours, once every six hours, once every seven hours, once every eight hours, once every nine hours, once every ten hours, once every eleven hours, once every twelve hours, once every thirteen hours, once every fourteen hours, once even fifteen hours, once every sixteen hours, once every seventeen hours, once every eighteen hours, once every nineteen hours, once every twenty hours, once every twenty one hours, once every twenty two hours, once every twenty three hours and once every twenty four hours.

78. The method of claim 42, wherein said patient takes said composition at a frequency selected from the group consisting of once every hour, once every two hours, once every three hours, once every four hours, once every five hours, once every six hours, once every seven hours, once every eight hours, once every nine hours, once every ten hours, once every eleven hours, once every twelve hours, once every thirteen hours, once every fourteen hours, once every fifteen hours, once every sixteen hours once every seventeen hours, once every eighteen hours once every nineteen hours, once every twenty hours, once every twenty one hours, once every twenty two hours, once every twenty three hours and once every twenty four hours.

79. The method of claim 42, wherein said composition is administered to said patient in a dose selected from the group consisting of 0.5, 1, 1.5,2,2.5,3, 3.5 and 4 dosage forms.

80. A composition consisting of butalbital, acetaminophen, caffeine and one or more inactive ingredients. wherein said composition is in the form of a gelcap.

81. The composition of claim 80, wherein said composition has about 25 mg to about 75 mg butalbital, about 200 mg to about 600 mg acetaminophen and about 20 mg to about 60 mg caffeine.

82. The composition of claim 81, wherein said composition has about 50 mg butalbital, about 400 mg acetaminophen, and about 40 mg caffeine.
